(19) United States
(12) Patent Application Publication
(10) Pub. No.: US 2025/0354222 A1
(43) Pub. Date: Nov. 20, 2025

(54) SENSOR, SYSTEMS, AND METHODS FOR DETECTING ENVIRONMENTAL DNA

(71) Applicant: Dartmouth Ocean Technologies Inc., Dartmouth (CA)

(72) Inventors: Vincent SIEBEN, Bedford (CA); Colin SONNICHSEN, Dartmouth (CA); Edward LUY, Halifax (CA); Andre HENDRICKS, Halifax (CA); Iain GRUNDKE, Halifax (CA); Aidan MACNEIL, Dartmouth (CA); Hayden EHLER, Dartmouth (CA); Kareem EL-BESHBEESHY, Dartmouth (CA); Carter HILTZ, Dartmouth (CA); Gabriel ROBERTS, Dartmouth (CA); Connor MACKIE, Dartmouth (CA); Mahtab TAVASOLI, Dartmouth (CA); Mark WRIGHT, Dartmouth (CA); Julie LAROCHE, Halifax (CA); Robert BEIKO, Upper Sackville (CA); Roger RACE, Dartmouth, MA (US); Tom KNOX, Fall River (CA); Jim HANLON, Tantallon (CA); Arnold FURLONG, Fall River (CA); James SMITH, Dartmouth (CA); Kyle Thomas PARK, Halifax (CA)

(21) Appl. No.: 19/207,823

(22) Filed: May 14, 2025

Related U.S. Application Data
(60) Provisional application No. 63/647,153, filed on May 14, 2024.

(51) Int. Cl. C12Q 1/6888; B01L 3/00; G01N 1/10

(52) U.S. Cl. CPC C12Q 1/6888; B01L 3/502715; B01L 3/502753; G01N 1/10; B01L 2200/027; B01L 2200/028; B01L 2200/0647; B01L 2300/0663; B01L 2300/0681; B01L 2400/0487; C12Q 2600/156; G01N 2001/1031

(57) ABSTRACT

An environmental DNA (eDNA) sensor and associated methods of detecting eDNA are disclosed. The eDNA sensor comprises a sample capture module configured to capture a fluid sample from a marine environment; a DNA extraction module fluidly coupled to the sample capture module and configured to extract and purify DNA captured by the sample capture module; and a genetic analysis module fluidly coupled to the DNA extraction module for analyzing the DNA to detect a target species. The eDNA sensor enables real-time, autonomous, and in-situ eDNA collection, extraction, purification, and detection. The sensor and methods can also be applied for environmental RNA (eRNA) detection.

### CROSS-REFERENCE TO RELATED APPLICATION

The present application claims priority to U.S. Provisional Patent Application No. 63/647,153, filed on May 14, 2024, which is incorporated by reference herein in its entirety.

### TECHNICAL FIELD

The present disclosure relates to sensing technologies for detecting environmental DNA and RNA in marine environments.

### BACKGROUND

Environmental DNA (eDNA) and RNA (eRNA) are emerging as a powerful complement to other technologies in the detection of organisms of interest. Traces of organisms left in the environment such as skin, urine, and feces contain DNA signatures of that individual organism, and several technologies exist to identify species based on these signatures.

One example use of eDNA detection is to protect marine mammals from harm and disturbance by underwater acoustic noise. When a marine mammal passes through an area it will shed skin cells and waste which contain DNA specific to that species. By collecting and filtering a volume of seawater, the shed organic material can be collected on a filter and analysed to enable detection of marine mammals. Detection through eDNA is independent of acoustic and visual signals and is specific to one or several target species. Moreover, inshore estimates indicate that eDNA may be detectable for approximately 48 hours, and even longer in offshore environments. However, one of the key challenges in using eDNA is the difficulty and expense of acquiring samples, which requires manual collection by highly trained personnel in the field and transport back to a laboratory for analysing. It is thus difficult, slow, and expensive to detect eDNA.

Accordingly, additional, alternative, and/or improved sensors, systems, and methods for detecting eDNA remains highly desirable.

### SUMMARY

In accordance with one aspect of the present disclosure, an environmental DNA (eDNA) sensor is disclosed, comprising: a sample capture module configured to capture a fluid sample from a marine environment; a DNA extraction module fluidly coupled to the sample capture module and configured to extract and purify DNA captured by the sample capture module; and a genetic analysis module fluidly coupled to the DNA extraction module for analyzing the DNA to detect a target species.

In some aspects, the eDNA sensor further comprises a microfluidic system configured to control fluid flow in the eDNA sensor and a controller configured to control the microfluidic system and to receive measurements from the genetic analysis module.

In some aspects, the DNA extraction module and/or genetic analysis module are implemented on a microfluidic chip.

In some aspects, the eDNA sensor further comprises a fluid storage section storing fluids used in the DNA extraction module and the genetic analysis module.

In some aspects, the sample capture module comprises a sample inlet port through which the fluid sample is received from the marine environment.

In some aspects, the sample capture module comprises one or more filter membranes for collection of cellular material from the fluid sample.

In some aspects, the sample capture module further comprises a pump for pumping the fluid sample across the one or more filter membranes for collection of the cellular material.

In some aspects, the sample capture module further comprises at least one of: a flow sensor configured to track a volume of fluid sampled, and a pressure sensor configured to track material loading on the one or more filter membranes.

In some aspects, the sample capture module is configured to capture and store a second fluid sample for archival.

In some aspects, the eDNA sensor comprises a sensor body housing the DNA extraction module and the genetic analysis module, and wherein the sample capture module is removably coupled to the sensor body.

In some aspects, the genetic analysis module performs quantitative polymerase chain reaction (qPCR) or DNA sequencing.

In some aspects, the target species is a marine mammal species.

In some aspects, the eDNA sensor further comprises a communication module configured to communicate a result of the genetic analysis module.

In some aspects, the eDNA sensor further comprises an on-board power module.

In accordance with another aspect of the present disclosure, an eDNA sensor system is disclosed, comprising a plurality of eDNA sensors of any one of the above aspects.

In accordance with another aspect of the present disclosure, a method of detecting environmental DNA (eDNA) is disclosed, comprising: capturing a fluid sample from a marine environment; automatically extracting DNA from the fluid sample; and analyzing the DNA to detect a presence of a target species.

In some aspects, the method further comprises detecting the presence of the target species, and communicating the presence of the target species to a remote device.

In some aspects, the method further comprises updating a density model for the target species based on the presence of the target species in the marine environment.

In accordance with the present disclosure, an environmental RNA (eRNA) sensor is disclosed, comprising: a sample capture module configured to capture a fluid sample from a marine environment; an RNA extraction module fluidly coupled to the sample capture module and configured to extract and purify RNA captured by the sample capture module; and a genetic analysis module fluidly coupled to the RNA extraction module for analyzing the RNA to detect a target species.

### BRIEF DESCRIPTION OF THE DRAWINGS

Further features and advantages of the present disclosure will become apparent from the following detailed description, taken in combination with the appended drawings, in which:

FIG. 1 shows a representation of an environmental DNA (eDNA) sensor architecture;

FIG. 2A shows a representation of an eDNA sensor in accordance with embodiments of the present disclosure;

FIG. 2B shows a cross-section of the eDNA sensor shown in FIG. 2A;

FIG. 3A shows a fluid schematic for the eDNA sensor in accordance with an embodiment;

FIG. 3B shows pressure recordings through a standard sampling protocol;

FIGS. 4A and 4B show fluid architecture diagrams of the eDNA sensor;

FIG. 5 shows a method of detecting environmental DNA (eDNA);

FIG. 6 shows a method of extracting eDNA from a fluid sample;

FIG. 7 shows an example of results from analysis of eDNA;

FIG. 8 shows a simplified system architecture diagram for the eDNA sensor;

FIGS. 9A-E show detailed system architecture diagrams for the eDNA sensor;

FIGS. 10A and 10B show mechanical drawings of the eDNA sensor;

FIG. 11 shows a representation of deployment of an eDNA sensor;

FIG. 12 shows a representation of a system of eDNA sensors;

FIG. 13 shows a temperature control diagram of a qPCR module;

FIG. 14 shows a representation of a heating manifold;

FIG. 15 shows a representation of a filter assembly;

FIG. 16 shows a representation of an eDNA sensor; and

FIG. 17 shows a fluid schematic for the eDNA sensor in accordance with an embodiment.

It will be noted that throughout the appended drawings, like features are identified by like reference numerals.

### DETAILED DESCRIPTION

The present disclosure describes sensors, sensor systems, and methods for detecting environmental DNA (eDNA) and environmental RNA (eRNA) from marine environments. The sensor described herein is an eDNA sensor that can collect and extract eDNA from a marine environment and analyze the eDNA for any sequences that are specific to target species such as marine mammals.

As described herein, the eDNA sensor comprises a sample capture module configured to capture a fluid sample from a marine environment; a DNA extraction module fluidly coupled to the sample capture module and configured to extract and purify DNA captured by the sample capture module; and a genetic analysis module fluidly coupled to the DNA extraction module for analyzing the DNA to detect a target species. An associated method of detecting eDNA (e.g. using the eDNA sensor) comprises capturing a fluid sample from a marine environment; automatically extracting DNA from the fluid sample; and analyzing the DNA to detect a presence of a target species. The sensor and methods described herein can also be applied for environmental RNA (eRNA) detection.

The sensors, sensor systems, and methods in accordance with the present disclosure enable real-time, autonomous, and in-situ eDNA collection, extraction, purification, and detection. The eDNA sensor reduces cost, increases throughput, and simplifies eDNA data collection, and furthermore enables remote, autonomous sensing for the marine environment. The eDNA sensor has a small form factor, and the automation allows for data collection over several months at remote locations.

With the eDNA sensor, environmental samples can be captured using the sample capture module by pumping a water sample from a marine environment over one or more filter membranes. The fluid samples may be captured on-demand, or autonomously at set periods of time. Shed cellular material that is collected on that filter is chemically treated to extract and purify the DNA contained in the sample. The purified DNA is analyzed to detect a target species. For example, the analysis can be used to produce a signal indicative of a marine mammal in the marine environment. The detected signal can be used to communicate information to a command center, nearby asset, etc., and used for decision-making (e.g. mammal avoidance), updating animal models (e.g. a density model that predicts marine mammal presence in an area), environmental monitoring, etc. By optimising the sub-routines of the sensor for speed and detection of target species, results can be obtained quickly (e.g. within an hour of sample collection). Actionable information can thus be provided. For example, in the case of marine mammals, information may be provided to avoid marine mammal ship strikes, acoustic damage, and/or disturbances to feeding, mating, and socializing behaviours.

As described above, the eDNA sensor comprises three modules or sub-systems that are fluidly coupled and operate in series: (1) a sample capture module configured to capture a fluid sample from a marine environment; (2) a DNA extraction module configured to extract and purify DNA captured by the sample capture module; and (3) a genetic analysis module for analyzing the DNA to detect a target species. Each of these modules present unique challenges for implementing in a compact and autonomous eDNA sensor with minimized turnaround time. As described in detail below, the eDNA sensor addresses and overcomes challenges such as contamination of the genetic material during sampling, potential presence of PCR inhibitors, choosing appropriate primers for the genetic analysis, precise fluid handling, temperature control in DNA extraction and analysis, etc. In accordance with some embodiments, the genetic analysis module performs quantitative polymerase chain reaction (qPCR). The qPCR process requires several changes in temperature at each amplification cycle, so the eDNA sensor is designed to accommodate rapid cooling and heating. Sampling a large volume of seawater can take a long time, especially with a small pump size that will be limited by the compact size of the eDNA sensor. Furthermore, several DNA extraction protocols take upwards of 90 minutes to extract and purify DNA, so there will be a limited set of protocols to work from and some optimization in protocol speed is required. The eDNA sensor in accordance with the present disclosure utilizes microfluidics and comprises a collection of valves, pumps, heaters, chemicals, sensors, optics, and onboard firmware in a compact space to control the fluid sample and to produce signals indicative of target species analyzed by the sensor.

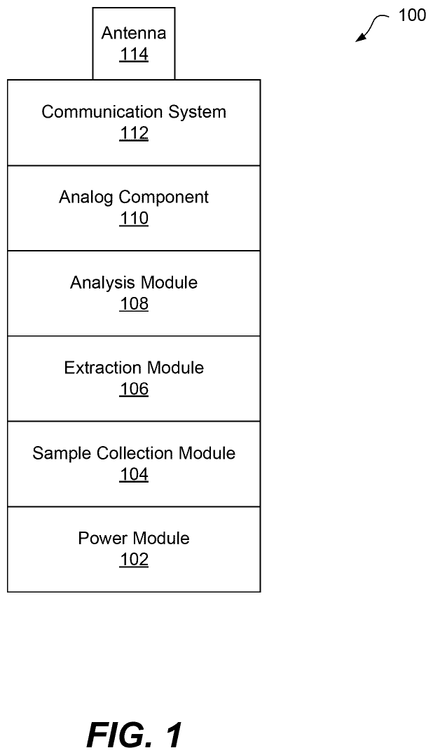

Embodiments are described below, by way of example only, with reference to FIGS. 1-12.

FIG. 1 shows a representation of an environmental DNA (eDNA) sensor architecture for an eDNA sensor 100. Specifically, FIG. 1 shows an overall representation of the eDNA sensor instrumentation, including the power supply, electronics, and communications that will enable sensor functionality, in accordance with embodiments of the present disclosure.

Power module 102 is configured to supply power to the eDNA sensor 100. The power module 102 may comprise on-board batteries to power the various systems including instrumentation within the sensor, and a PCB to manage that power and handle battery charging and discharging safely.

The sample collection module 104 is configured to capture a fluid sample. The sample capture module may comprise a sample inlet port through which the fluid sample is received from the marine environment. In some embodiments, the sample capture module comprises at least one filter membrane for collection of cellular material from the environment. The sample collection module 104 may also comprise at least one pump for pumping the fluid sample across the filter membrane(s) for collection of such material. A flow sensor may be provided in the sample collection module 104 to track volume of fluid sampled, and a pressure sensor may be used to track material loading on the filter membrane(s).

The extraction module 106 is configured to extract and purify DNA captured by the sample capture module 104. The extraction module 106 may comprise a solid phase column that will bind DNA in a high salt solution, allowing contaminants to be washed off the DNA. Additional reagents may be present in the extraction module 106, and the extraction module 106 may also include additional elements such as a heating element, etc.

The analysis module 108 is configured to analyze the DNA to detect a target species. The analysis module 108 comprises various equipment for performing genetic analysis, which may in particular be quantitative polymerase chain reaction (qPCR) analysis, although other eDNA analysis methods may be used, such as eDNA sequencing. The analysis module 108 may comprise a heater, optical sources, filters, and detectors, as well as the reagents for DNA analysis. The analysis module 108 may also comprise reagents for sterilization of the instrument's internal surfaces.

Analog component 110 comprises a controller, such as a printed circuit board that controls the various components and instrumentation of the eDNA sensor 100, such as the various pumps, motors, valves, sensors, heaters, and communications within the instrument. The analog component 110 may also provide data from the analysis module 108 to the communication system 112 for external communication.

The communication system 112 is configured to receive and log results and prepare the results for telemetry. The communication system 112 may also receive data from an external device and provide such data to the analog component 110 to develop actions to be performed by the eDNA sensor 100.

An antenna 114 (e.g. RF or satellite) may be provided for remote communication and data retrieval.

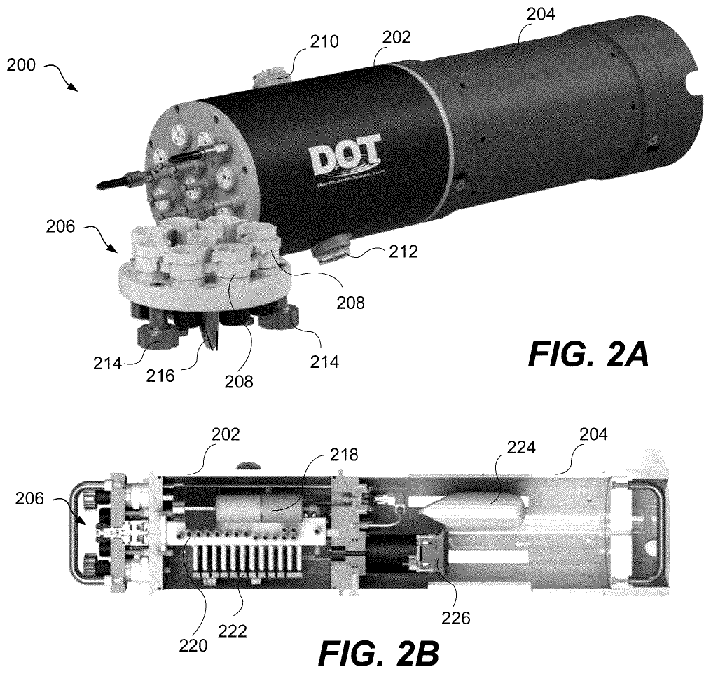

FIG. 2A shows a representation of an eDNA sensor 200 in accordance with embodiments of the present disclosure. FIG. 2B shows a cross-section of the eDNA sensor 200 shown in FIG. 2A. The eDNA sensor 200 comprises the architecture of the eDNA sensor 100 described with reference to FIG. 1. The eDNA sensor 200 is a standalone sampling device that is compact and fully autonomous.

The eDNA sensor 200 is an autonomous instrument capable of collecting, filtering, preserving, and analyzing a water sample using a compact and innovative design as shown in FIGS. 2A and 2B. The eDNA sensor 200 comprises a sampler body 202, which performs the major sampling functions (electronics, logging, automation, etc.) and contains microfluidic piping and instrumentation (fluid pumps, valves, paths, etc.). The eDNA sensor 200 further comprises a reagent housing 204, which is a perforated shell containing fluid reservoirs (in this embodiment, four fluid reservoirs) storing fluid used for DNA extraction and analysis. The eDNA sensor 200 also comprises a filter cassette 206, comprising filter membranes 208 (in this embodiment, nine filter membranes) for collecting discrete samples per deployment. A sample inlet port 210 is used for drawing sample fluid, and a sample outlet port 212 is for sample and cleaning cycle waste. The filter cassette 206 is removable and can be easily and rapidly changed on site. This removable cassette allows for immediate redeployment of the instrument, where a new cassette can rapidly be loaded, and the full cassette is either analyzed in the field or back at the lab. The filter cassette 206 may comprise cassette knobs 214 to attach or release the filter cassette 206 to/from the sampler body 202, a handle 216 for transport, etc. Furthermore, the eDNA sensor 200 is self-cleaning to prevent biofouling, and all tubing is contained within the instrument housing to prevent snags on the lines during deployments. The sensor is also compact and designed with dual handles, allowing it to be transported and deployed by a single person.

The eDNA sensor 200 features a simple modular approach such that each of the filter cassette 206; electronics section (sampler body 202); and fluid storage section (reagent housing 204) can be detachable, as shown in FIGS. 2A and 2B. The eDNA sensor's filter cassette 206 may be made from a hard plastic material and secures the filter holders. Once the filter cassette 206 is loaded with clean filters (filter membranes 208), it can be attached to the electronics section of the eDNA sensor. This fast swap approach allows for multiple filter cassettes to be prepared and then loaded into the sensor as needed. The filter cassette may be secured by three knobs 214 that are indexed to the electronic section to avoid assembly error. The sensor 200 may be configured for deep deployments (e.g. up to 3000 meters).

The eDNA sensor's electronics section is the core of the instrument. The electronics section houses a pump 218 (e.g., a syringe pump) and custom valve tree 220 (e.g., a solenoid valve tree), along with a custom printed circuit board (PCB) 222 for automation and data logging. The PCB 222 regulates voltage to the various electric modules, controls the valves and pump, logs pressure data, and controls sampling schedules. The valve tree consists of the fluid routing manifold, a pressure sensor, tubing inter-connections, and solenoid valves for the sensor. The valve tree 220 also has ports that are used to fluidically couple to the filters (filter membranes 208) on the filter cassette 206, and to access the fluid bags 224 loaded with reagents and stored in the fluid storage section (reagent housing 204) of the sensor.

The fluid storage section houses and protects all the required fluids and an optional fluorometer 226. The fluids stored in this section may be as follows: 5% hydrochloric acid (HCl) (cleaning), RNAlater (preservation), purified Milli-Q water (rinsing), and waste. The fluids may be stored in 100 mL and 500 mL sterile, semi-rigid bags and connected to the electronics section through threaded fluid ports. A waste bag may be used to hold chemicals that are not safe to flush into the ocean or surrounding waters. RNAlater is used to preserve the collected samples, 5% HCl is used to prevent genetic contamination by cleaning the common fluid lines and flowing backwards across the sample inlet. Milli-Q is used to flush previous samples, HCl and RNAlater from the system between protocol steps. The 5% HCl and Milli-Q are effective at reducing cross-contamination that might take place in the system tubing and manifolds between sampling events.

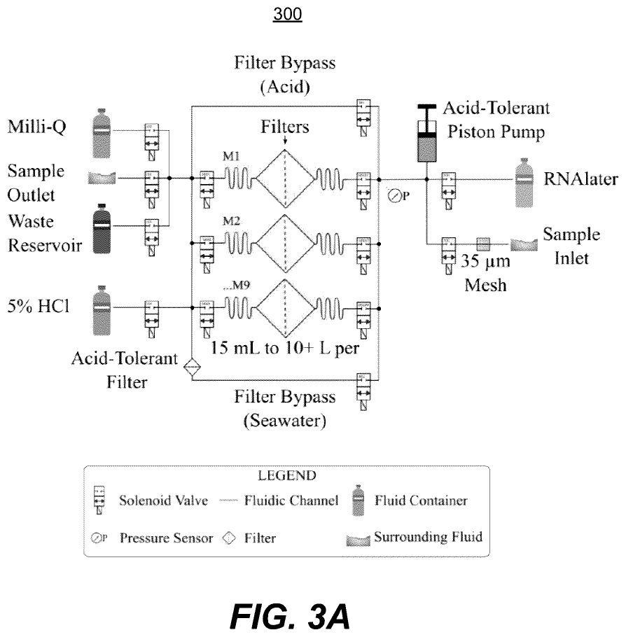

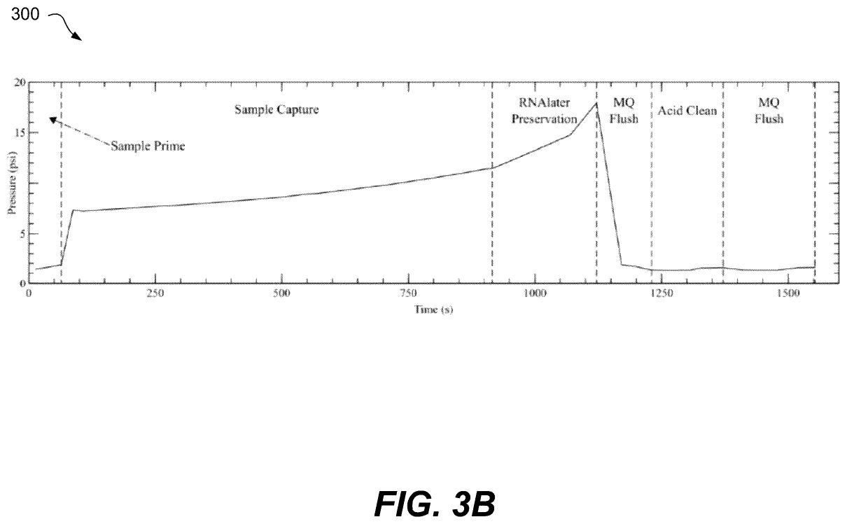

FIG. 3A shows a fluid schematic for the eDNA sensor in accordance with an embodiment. The eDNA sensor features several solenoid valves, filter membranes, onboard chemicals, and access to the surrounding fluids via the sample inlet and outlet ports. The eDNA sensor features custom control scripts that can be used to coordinate operations between the solenoid valves and syringe pump in any conceivable configuration. This flexibility allows easy adaptation to future designs or systems; for example, to control an eDNA sensor. The movement of fluid is performed in concurrence with the monitoring and logging of both fluorometer and pressure readings. The fluorometer is a useful accessory for eDNA sampling because a fluorometer can monitor chlorophyll abundance in the environment. If the fluorometer detects an increase in chlorophyll, it could be used to trigger an eDNA sample. For marine mammal eDNA, the external fluorometer is not necessary. Custom sampling protocols can be written by the user and uploaded to the sensor's SD card storage via serial communication or through the eDNA sensor software.

FIG. 3B shows a graph 310 of pressure recordings through a standard sampling protocol.

The following protocol was used to perform the initial field testing of the eDNA sensor. The pressure sensor reading throughout this protocol is shown graphically in FIG. 3B. The protocol is broken up into six sequential phases. The “Sample Prime” step commences the sampling protocol and prepares the sensor by flushing its internal channels with the environmental sample. Thereafter, the sensor is now ready to perform the “Sample Capture” step. This step pushes the sample fluid through the selected filter membrane (M1 through M9) for sample capture. To preserve the material collected on the filter, the “RNAlater Preservation” step pushes the RNAlater through the selected filter membrane. The “MQ Flush” step then uses Milli-Q to flush RNAlater from the system. The “Acid Clean” step cleans and sterilises the sensor's internal common fluid channels of contaminants using 5% HCl. Finally, the “MQ Flush” step flushes the 5% HCl from the channels using Milli-Q. This process cleans the sensor and prepares it for the next sample capture.

An adaptive flow-rate algorithm may be used to filter samples as quickly as possible without building up excessive pressure. An internal pressure sensor may be used to monitor the internal pressure of the system during sampling. If the measured pressure rises above a specified pressure threshold, then the sampling flow rate is decreased before pumping resumes. This loop continues until either the full sample capture volume is pumped, or until the flow rate drops below a minimum value. When the latter occurs, the rest of the protocol continues, and the filtered volume is recorded for the user. When not sampling, the sensor enters a low-power state and waits for an interruption to trigger the sampling protocol once more.

The sensor is fully autonomous; once a schedule is set the sensor will perform all functionalities without the need for user input. The only human interaction is to change the filter cassette, chemicals, and battery in addition to programming the scheduler. Beyond scheduled triggering, the eDNA sensor may comprise an onboard 32-bit processor that allows samples to be triggered by external sensors and computers (e.g. AUV backseat systems). In this regard, the eDNA Sensor is highly adaptive to various “smart sampling” triggers. For example, a sample capture can be triggered based on measured environmental conditions such as temperature, turbidity, or external pressure. Otherwise, samples can be captured through more conventional means, either from wire activation or using a pre-programmed date and time schedules.

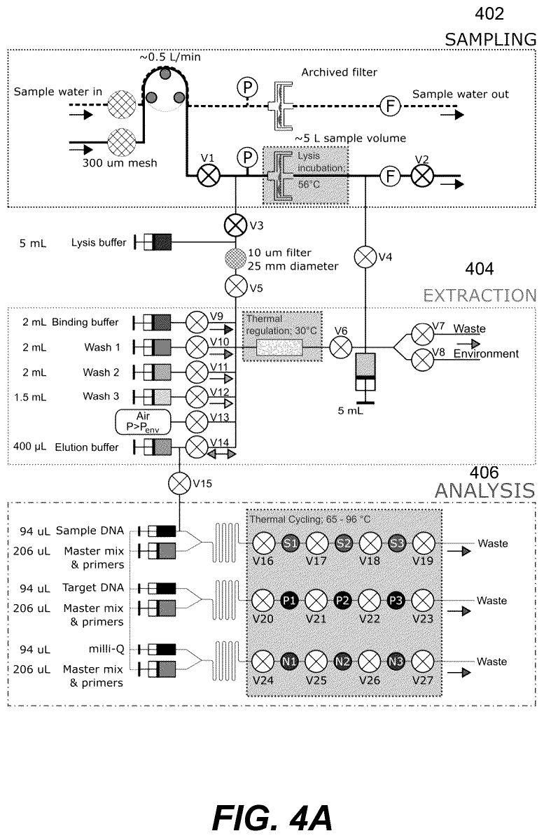

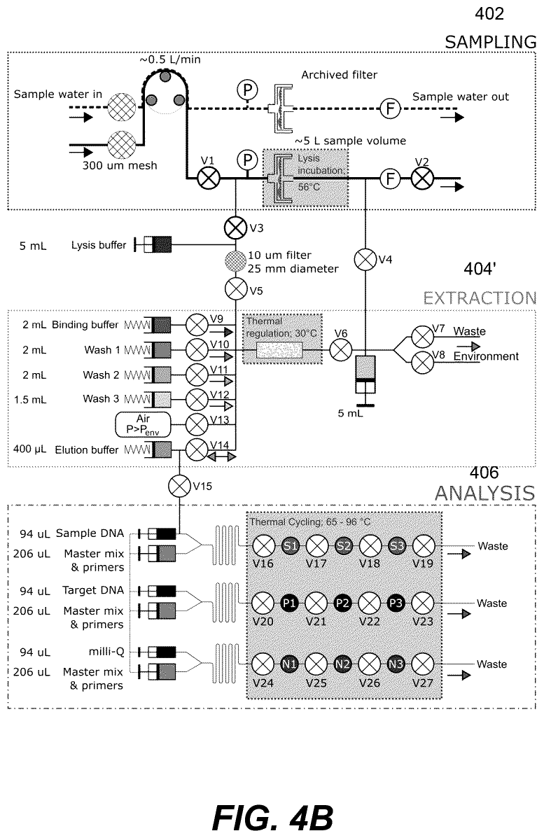

FIGS. 4A and 4B show fluid architecture diagrams of the eDNA sensor, which as described above is capable of autonomously filtering seawater, extracting DNA from that filter, and amplifying said DNA for analysis/detection. While FIGS. 4A and 4B show specific architecture diagrams, the general components include: at least one filter for sample collection; a number of pumps and valves to control flow; a solid phase extraction column; a collection of reagents for DNA extraction and analysis; hating elements; optical elements; tubing; a flow meter; and a pressure sensor. To reduce the size and complexity of the eDNA sensor, aspects of the fluid architecture may be implemented on a microfluidic lab-on-a-chip (LOC). For example, a microfluidic LOC device could integrate extraction and qPCR in one small device, simultaneously enabling low cost eDNA sensing and rapid detection of target DNA.

As described above, the eDNA sensor comprises three primary modules: a sampling module 402, an extraction module 404, and an analysis module 406. The fluid architectures shown in FIGS. 4A and 4B are substantially the same, however in FIG. 4A actuators are used in the extraction section 404, while in FIG. 4B springs are used in the extraction section 404′.

Samples are drawn from the environment in the sampling section 402. A pump draws a sample fluid in from the environment and pushes it across a filter to capture organic material. The volume sampled is measured with a flowmeter. The amount of material on the filter is monitored with the pressure sensor. Once a target volume has been sampled or the pressure reaches a threshold value, a second capture event may start. The second filter can be used for lab analysis and may not be analysed onboard the instrument, thus allowing for subsequent validation of the analysis result. Fluid direction may be controlled through solenoid valves at the inlet and outlet of the sampling section. Syring pumps may be used due to their precise control over fluid volumes, small size, and long lifetimes. Alternatively, a single sample eDNA sensor may use a peristaltic pump for increased sampling speed, and where precise control over volume is not necessary.

Once both filters have captured material, the material on one of the filters can be treated with a chemical lysing agent to break open cell walls. The lysed cells can then be pumped to the extraction section. The extraction section 404, 404′ is fluidly coupled to the sampling section 402 and configured to extract and purify the DNA captured by the sampling section by pumping the lysed cells over a solid phase extraction column, where DNA will bind to the column, and cleaning the DNA with another series of chemicals before it is transferred to the analysis module. Various pumps and valves are used for precise control over the small volumes used in extraction (<1 mL). Pumps may also be used for back-flowing over the filter to remove material without sending lysed cells towards waste. The valves may be used to control the direction of flow through the filter and extraction column. Once DNA is extracted and purified, an elution buffer is flowed over the filter and extraction column to put the DNA into solution.

The DNA-rich solution that comes out of the extraction module is pushed into the analysis section 406. For implementation in an in-situ eDNA sensor, the analysis section must be small, light, and portable to fit within the body of the sensor, and the time to run the analysis must be reasonable for near real-time detection. The analysis section may be configured to perform qPCR analysis. Once DNA from the extraction module is pumped into the analysis section, the sampled DNA is mixed with another set of reagents in a thermally cycled compartment, and a thermal cycling procedure can begin. After each thermal cycle fluorescent counts are measured. If DNA that is complementary to the primers is present, that DNA gets amplified exponentially at each cycle, and the fluorescent signal grows in proportion. By detecting the exponential growth in fluorescence in real-time, the positive detection of target DNA is inferred and the prototype sensor outputs a positive result.

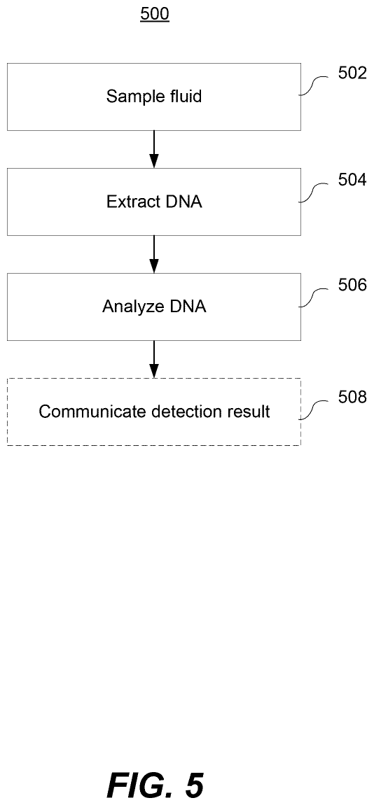

FIG. 5 shows a method 500 of detecting environmental DNA (eDNA). The method 500 is performed by the eDNA sensor described above, e.g. under the operation of the controller.

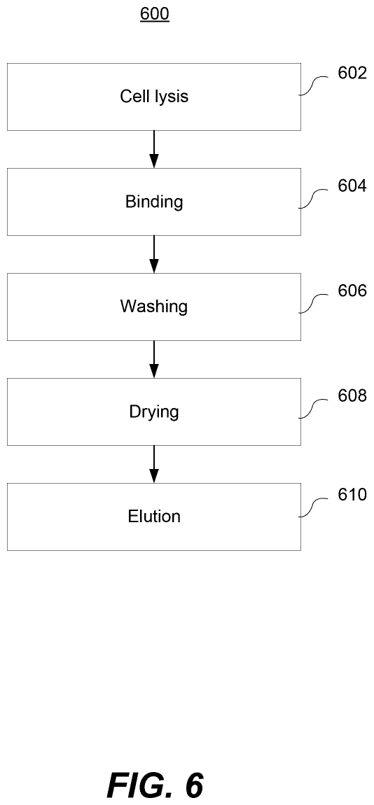

The method 500 comprises capturing a fluid sample from a marine environment (502), e.g. using a sample collection module as described above. DNA is automatically extracted from the fluid sample (504), e.g. using a DNA extraction module as described above. A method of extracting eDNA from a fluid sample is described further with reference to FIG. 6. The DNA is analyzed to detect a presence of a target species (506). In some aspects, the method 500 detects presence of the target species, and the method 500 may further comprise communicating the presence of the target species to a remote device (508). Additionally or alternatively, a density model for the target species may be updated based on the presence of the target species in the marine environment.

FIG. 6 shows a method 600 of extracting eDNA from a fluid sample. The method 600 is performed by the eDNA sensor described above, e.g. under the operation of the controller.

The method 600 is used to perform solid phase extraction of DNA from a collection of cellular material, and comprises performing the following: Cell lysis (602), which comprises destroying cell walls and freeing DNA from the cell; Binding (604), which is when the DNA attaches to the solid phase while cell debris floats freely; Washing (606), which comprises destroying cellular proteins and enzymes, and removing PCR inhibitors; Drying (608), which comprises forcing air over the solid phase to purify the DNA and remove any liquid; and Elution (610), which comprises using a low-salt solution to release and collect the purified DNA from the solid phase.

The Biomeme M1 sample prep cartridge DNA extraction protocol was used as the basis for implementing the method 600 in the eDNA sensor, which was found to be most adaptable to small, autonomous platforms for use in the field. The method 600 may comprise variations in the choice of reagents used, incubation times and temperatures, or the nature of the binding material. The eDNA sensor is designed through a collection of tubing, valves, pumps, and heaters to implement the method 600 through control by a controller (e.g. microprocessor).

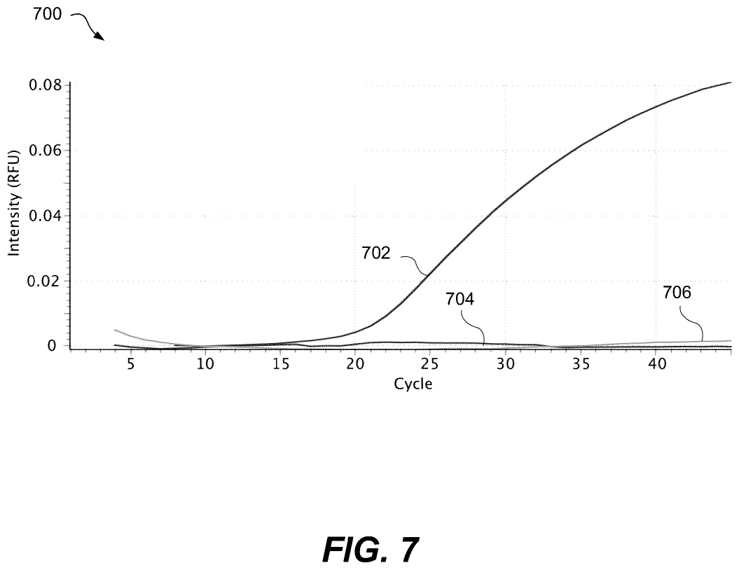

FIG. 7 shows an example of results from analysis of eDNA. As described with reference to the method 500, DNA is analyzed to detect a presence of a target species.

In this example, genetic analysis was performed through a quantitative PCR (qPCR) assay specific for whales. Polymerase chain reaction (PCR) is an enzymatic process that makes copies of a specific DNA sequence found in a sample, which can then be detected after a number of amplification cycles. Cycling is controlled by temperature in the PCR reaction chamber. The amplification is specific to a target segment of DNA, determined by a pair of DNA primers. The quantity of target DNA grows exponentially with the cycle number. The difference between qPCR and PCR is the addition of a fluorescent dye to the PCR mixture, which emits an optical signal at every PCR cycle to track the progress of amplification. The fluorescent signal allows the amplification to be quantified at each cycle relative to a calibration standard, hence the term quantitative PCR. Typically, if the fluorescent signal crosses a threshold before a set number of amplification cycles it results in a positive detection of the target DNA. qPCR also allows an early detection of the targeted DNA such that a higher concentration of the target DNA leads to an earlier crossing of the fluorescent signal threshold, which allows for a determination of DNA concentration in the original sample.

The fluorescent signal in qPCR can come from two kinds of fluorescent molecules: Ones that are activated by binding to double stranded DNA, or ones that are activated when DNA gets amplified. The latter have a short DNA ‘probe’ segment that binds to the target DNA, so they are specific to the target species.

The graph 700 shows the results after qPCR analysis using a MyGo benchtop qPCR instrument for initial testing and proof of concept. The graph 700 demonstrates that bottlenose whale DNA was successfully amplified and detected in eDNA medium (line 702), and that no other DNA products were amplified (e.g., line 704 shows eDNA medium without bottlenose whale DNA, and 706 shows no DNA data).

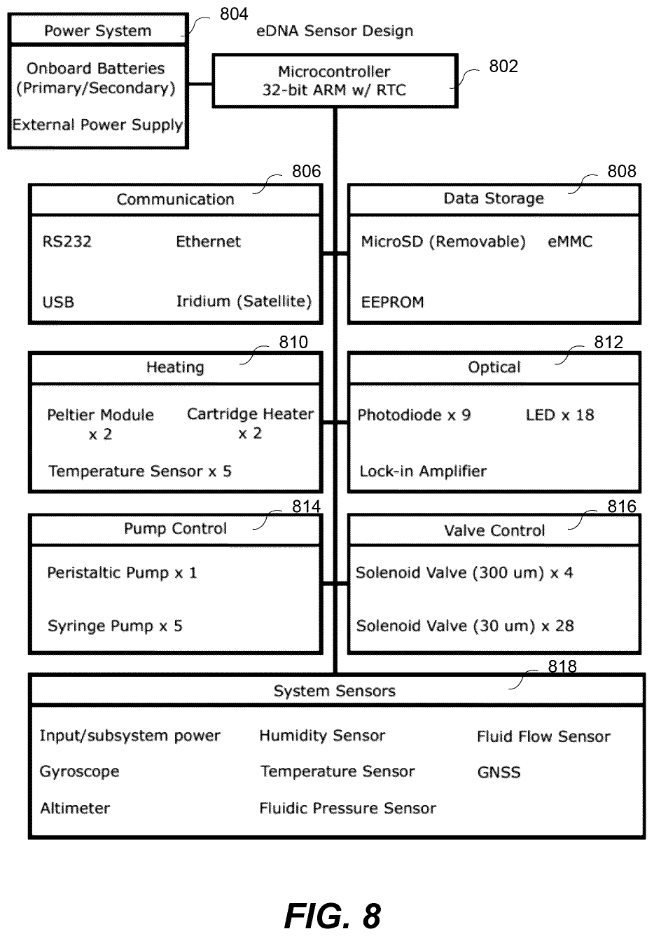

FIG. 8 shows a simplified system architecture diagram for the eDNA sensor. The eDNA sensor comprises a microcontroller 802, which receives power from a power system 804. The power system 804 may comprise onboard batteries or be an external power supply. The microcontroller 802 is further coupled to a communication module 806, a data storage module 808, a heating module 810, an optical module 812, a pump control module 814, a valve control module 816, and system sensors 818.

The communication module 806 may support RS232, USB, Ethernet, and/or Iridium (satellite) communications. The data storage module 808 may comprise a removable MicroSD, an eMMC, and/or EEPROM. Heating module 810 may comprise Peltier module(s), cartridge heater(s), and various temperature sensors. Optical module 812 may comprise photodiodes, LEDs, and a lock-in amplifier. Pump control 814 may comprise a peristaltic pump and syringe pumps. Valve control module 816 may comprise various solenoid valves. System sensors 818 may comprise input/subsystem power, gyroscope, altimeter, humidity sensor, temperature sensor, fluidic pressure sensor, fluid flow sensor, GNSS, etc.

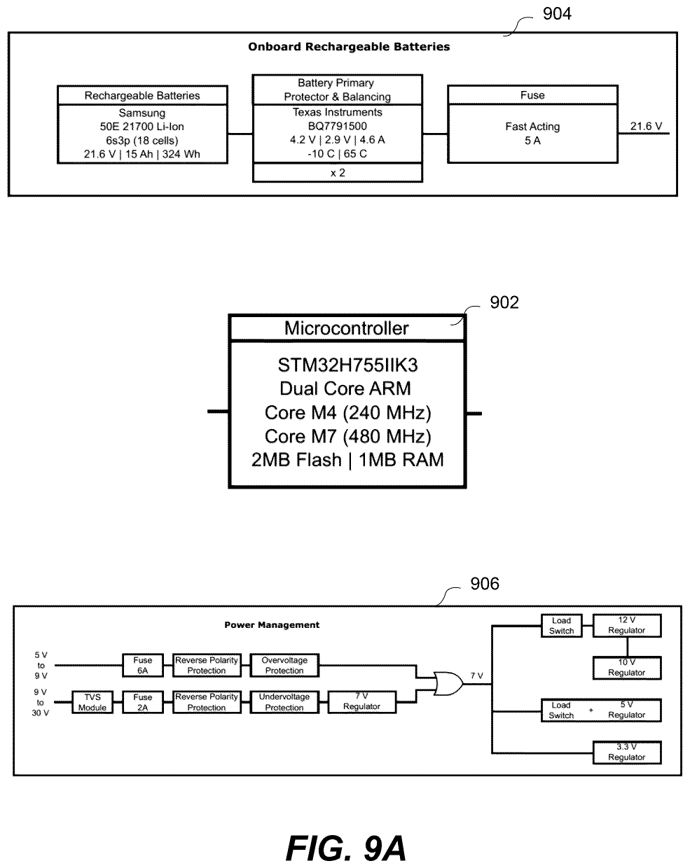

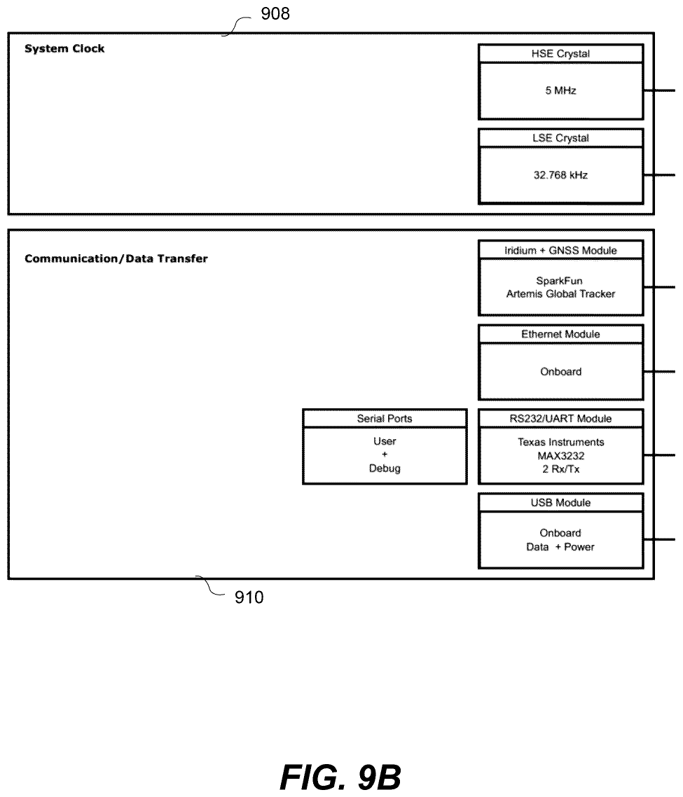

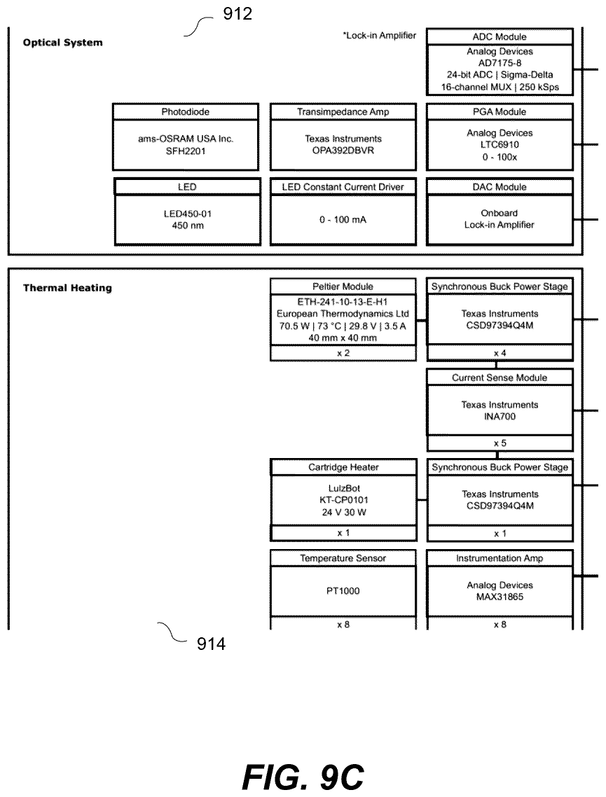

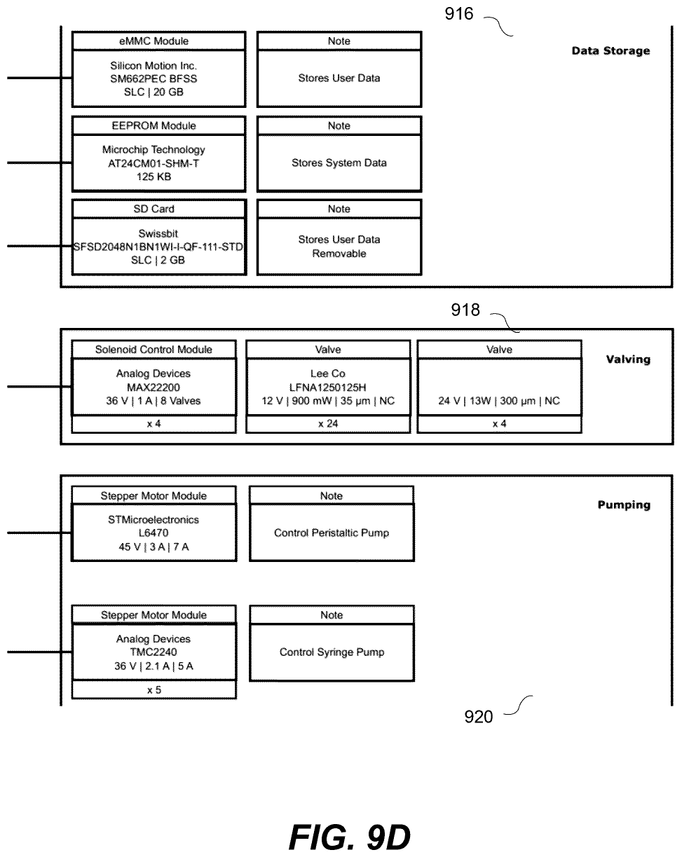

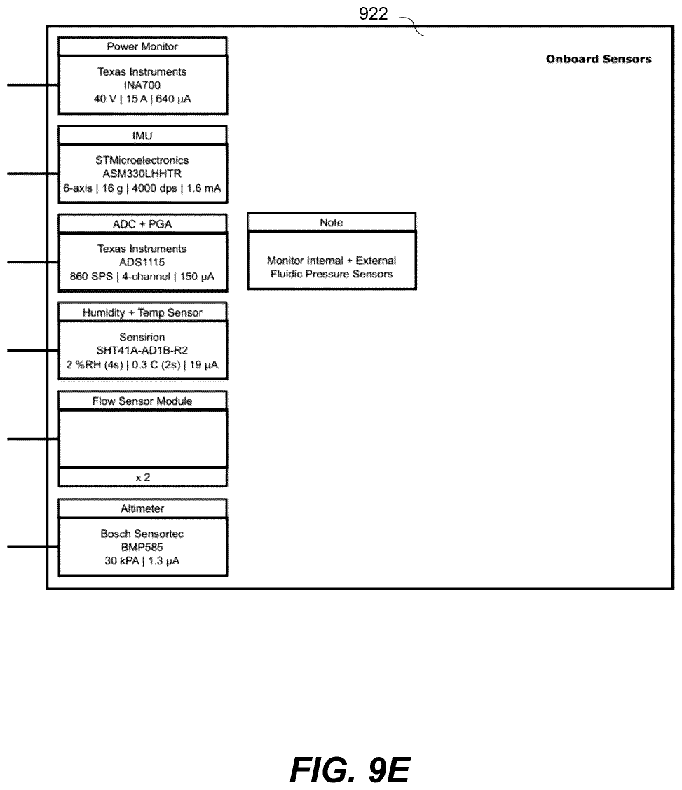

FIGS. 9A-E show detailed system architecture diagrams for the eDNA sensor. A microcontroller 902 is used to control the eDNA sensor. Onboard rechargeable batteries 904 provide power to the eDNA sensor, and a power management system 906 comprises various fuses, switches, and regulators to manage the power in the eDNA sensor and provide overvoltage and undervoltage protection. The eDNA sensor also comprises a system clock 908, a communication/data transfer module 910, an optical system module 912, a thermal heating module 914, a data storage module 916, a valving module 918, a pumping module 920, and an onboard sensor module 922 as shown.

Electrical control may thus be provided by a circuit board that generally has the following components: microcontroller with real-time clock; fluorescence module for qPCR; temperature controller for qPCR and extraction; fluid control module for control over pumps and valves; voltage regulation from a battery to the various devices on the eDNA sensor; and communications to the outside world via RF, IRIDIUM and GPS localisation.

An activation signal is sent to the microcontroller, which communicates with the various subcomponents of the eDNA sensor. For example, a pump driver may be used to read home switches, encoders, and supply current to at least two pumps. A valve driver may be used to send signals and power to the bank of valves to control fluid flow. The fluorescence module is used for qPCR, since DNA amplification is detected through fluorescence. A short-wavelength LED may be used to stimulate fluorescence and a photodetector to monitor the long-wavelength signal. The temperature control module with a heater and thermistor is used for controlling temperature in the qPCR chamber and also in the extraction module. Power is supplied by an onboard battery pack. Activation may be implemented with a mechanical switch, that might be triggered by a set date and time or an RF signal.

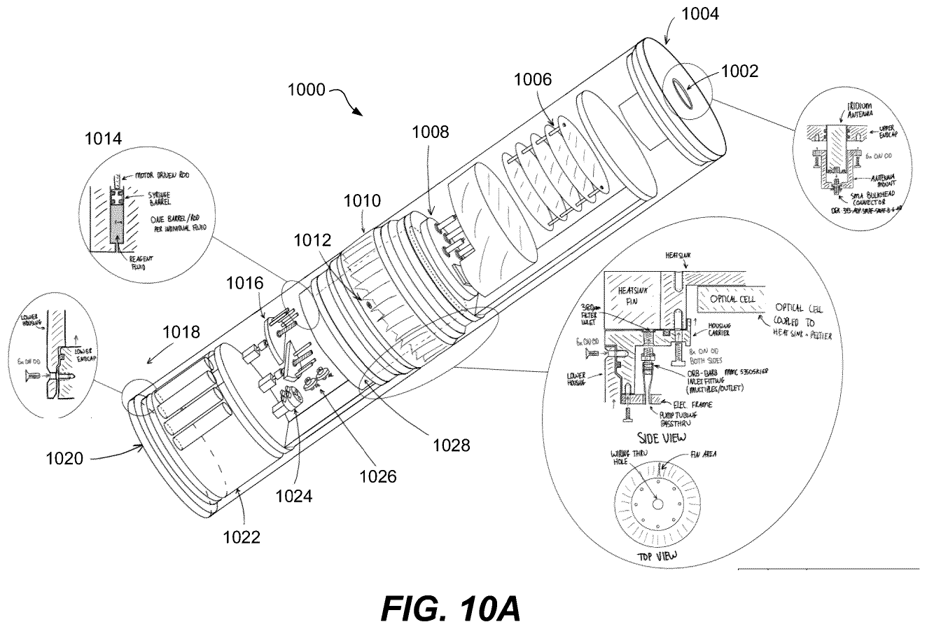

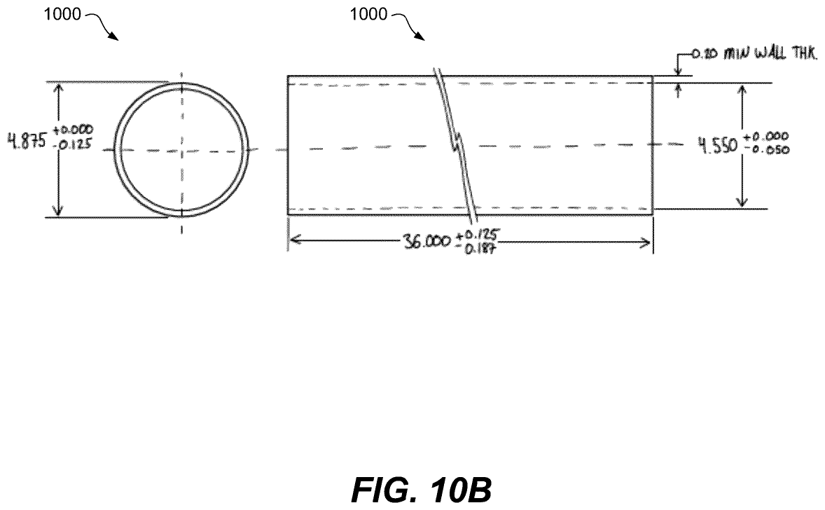

FIGS. 10A and 10B show mechanical drawings of the eDNA sensor 1000. FIG. 10A shows a transparent perspective view, and FIG. 10B shows a side view and an end view of the eDNA sensor 1000. In this exemplary design, the eDNA sensor is configured as an A size sonobuoy. It will be appreciated that the design shown in FIGS. 10A and 10B is provided solely for the sake of example, and that all dimensions are non-limiting. Is should be noted that the dimensions shown in FIG. 10B are for illustration purposes only and should not be construed as limiting the scope of this application.

As shown in FIG. 10A, the eDNA sensor comprises an antenna 1002 (e.g. Iridium antenna) for data communication; provisions 1004 for air descent control system and decelerator; an electrical frame 1006 comprising PCBs and other electronics for electrical and optical control; microfluidic system 1008 for fluid handling and optical interface with thermocycling Peltier heater and qPCR optical cells; a heatsink 1010, made for example of anodized aluminum; a fluid inlet 1012, which may comprise a filter mesh through the heatsink to help with cooling and clogging; a cartridge system 1014, comprising a puck with individual finger operated plungers and wherein syringe volumes are determined by plunger diameter, two syringe banks, for extraction and analysis respectively, and varied radii for targeting volume with constant stroke microfluidic delivery channels; a pumping mechanism 1016; a split housing, with separated upper/lower compartments, where the lower housing may slide off as shown at 1018 to change/service fluids cartridge, replace used seawater filters, and charge/service battery; a lower endcap 1020, as shown in detail; a battery pack 1022, which may comprise a plurality of lithium cells; a dual-headed peristaltic pump 1024; seawater filters 1026; and a reagent cartridge 1028, which contains all fluids required for DNA extraction and analysis and may be removable.

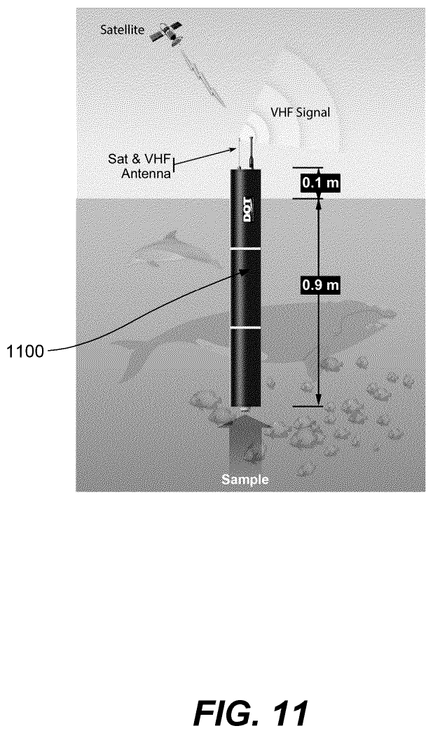

FIG. 11 shows a representation of deployment of an eDNA sensor 1100. As seen in FIG. 11, the eDNA sensor 1100 is integrated in a standard “A”-size sonobuoy form factor to enable deployment. It should be noted that the depths shown in FIG. 11 are for illustration purposes only and should not be construed as limiting the scope of this application.

The eDNA sensor is a fully autonomous, in-situ device for sampling, extracting, and analyzing DNA found in the environment. Samples may be collected by pumping seawater over a filter at a set time, allowing for manual deployment followed by remote sensing. The shed cellular material that is collected on that filter is chemically treated to extract and purify the DNA contained in the sample. The purified DNA is injected into an analysis module, such as a qPCR device, which will amplify only the target DNA sequence of marine mammals. The amplified DNA will produce a signal that can be detected, indicating the presence or absence of target DNA in the area. This information is sent back to a command center through satellite communications or to a nearby asset via line-of-sight VHF-FM radio. This described system allows for remote monitoring of marine mammals in near real-time, with no human interaction.

The eDNA sensor thus provides a simple, single-person portable, modular device. The entire sensor package may be targeted to an A-size sonobuoy form factor. The sensors can be shipped with most of the reagents and chemistry pre-loaded and primed. However, the primers and reagents for qPCR analysis must be stored in the dark and refrigerated. Therefore, the first step in using the eDNA sensor is to retrieve reagents from storage and load them into the sensor. The sensor is designed to make this a user-friendly, intuitive procedure.

After loading reagents, the sensor may be deployed in the environment by dropping it off the side or stern of a ship. It may also be suitable for deployment from rotary or fixed-wing aircraft. Sampling can begin through a pre-programmed set time or RF signal. Once the sensor is triggered, a preset volume of seawater will be pumped over the sample capture filter. Once sample capture is complete, the DNA is extracted, purified, and analyzed through qPCR. The number of PCR heating cycles before the detection threshold is reached, along with sample location, GPS coordinates, and qPCR primer ID, is sent back to a central controller through telemetry. The number of heating cycles is an important measure that relates back to the initial quantity of DNA in the environment, fewer heating cycles means more DNA was likely present at the time of sampling. Telemetry may be either RF broadcast or Iridium satellite communications. Once sensing is complete the instrument can be recovered through GPS localization. If the sensor will be re-used it must be sterilized and a new chemical cartridge and batteries must be loaded. Alternatively, the eDNA sensor can be left to sink to the seafloor.

As shown in FIG. 11, each of the three sub-systems of the eDNA sensor may be mounted vertically. A reagent cartridge is loaded into the top qPCR section of the device before deployment. The center of gravity of the sensor is towards the sampling section, such that when deployed the sensor floats with the sampling portion facing the seafloor. Sample intake is from the bottom face of the sensor. The location of the center of gravity also guarantees that the RF and satellite communications are above the waterline. Power is supplied through an onboard battery pack, so that no external cables or pressure cases are necessary. Where the instrument is used as a single sample device with short operation times, power consumption will be relatively small.

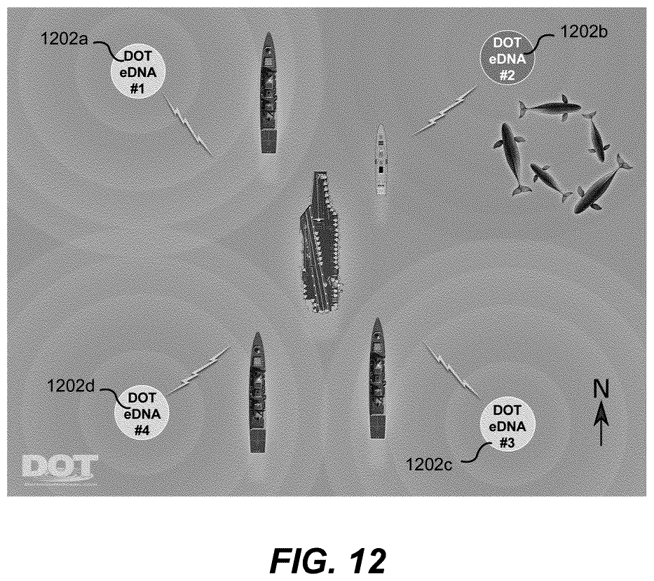

FIG. 12 shows a representation of a system of eDNA sensors, 1202a-d. It will be appreciated that for some implementations, a network of eDNA sensors may be deployed. For example, based on the data received from four to five sensors (presence/absence of target DNA, GPS coordinates and sampling times) and in conjunction with knowledge about the ocean conditions (wind speed and direction, sea state, ocean currents, etc.) a likelihood of marine mammal presence in the area can be modeled. The probability map that is constructed would evolve in time as new data comes in from other eDNA sensors, visual sightings, or acoustic monitoring.

All four sensors sample the ocean environment at their respective locations, and report back to the exercise command center. In this example, eDNA sensor #2 (1202b) has detected whale DNA, so is shaded in FIG. 12.

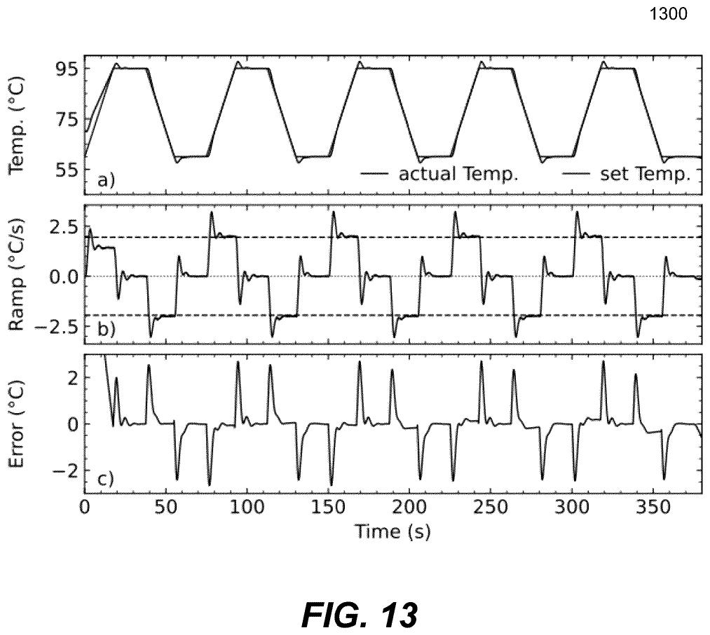

In some embodiments, temperature of a qPCR module may be controlled by a regulating device, such as heat sink, a Prltier device, combination thereof, or other suitable devices. FIG. 13 illustrates an example of temperature control diagram 1300 of the qPCR module.

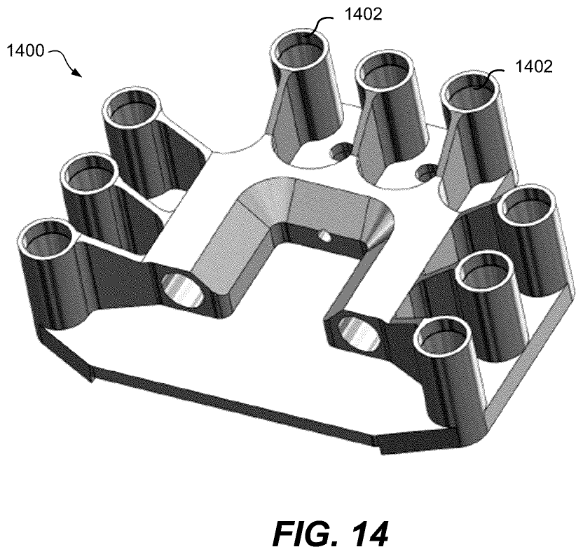

FIG. 14 illustrates an example of a heating manifold 1400 for a qPCR module. The heating manifold 1400 may include multiple receptacles or qPCR tubes 1402 for performing eDNA sensing events, and may have high surface area to volume ratio to enable rapid thermal cycling. The heating manifold 1400 may be made of a material that has high thermal conductivity, such as aluminum for enhanced thermal transfer properties.

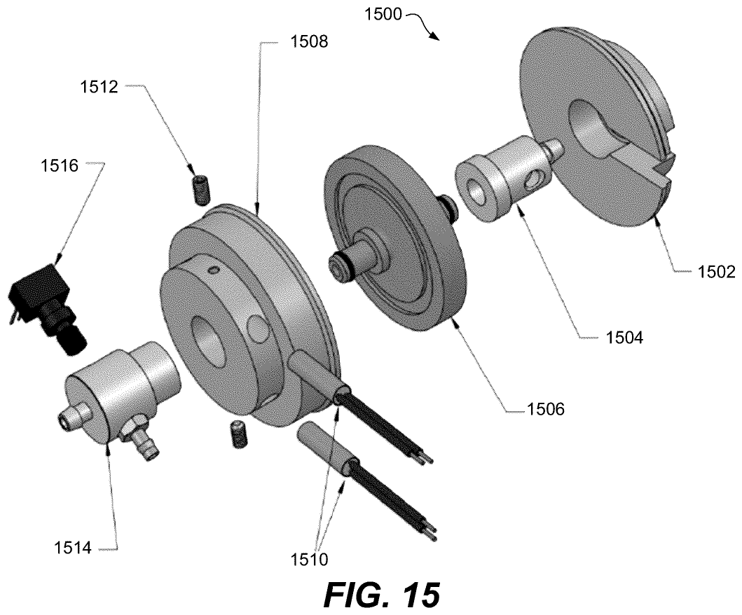

FIG. 15 illustrates an example of a sampling filter assembly 1500, which includes an outlet side heater core 1502, an outlet side filter adapter 1504, a filter 1506, an inlet side heater core 1508, multiple heating elements 1510, multiple heating element set screws 1512, an inlet side filter adapter 1514, and a pressure sensor 1516.

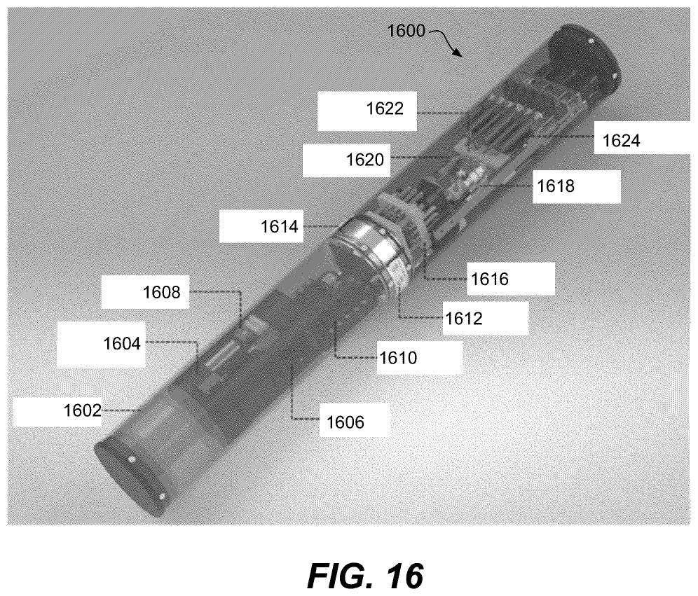

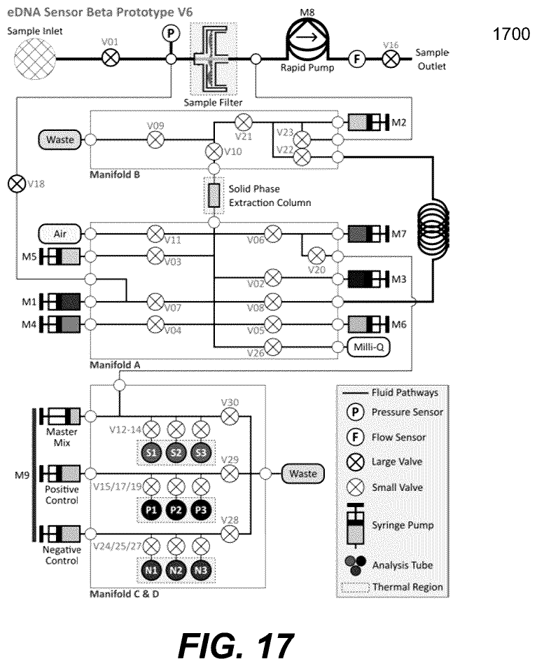

FIG. 16 illustrates an example of an eDNA sensor 1600, which is similar in structure and function as the eDNA sensor 200 of FIGS. 2A-B. The eDNA sensor 1600 includes a power supply 1602 (e.g., a Li-ion battery), a pump 1604 (e.g., a diaphragm pump), a first PCB 1606 (e.g., a main PCB), a second PCB 1608 (e.g., an Iridium PCB), an optical array 1610, a sample inlet 1612, a heat sink 1614, a qPCR module 1616, a sample filer 1618, a solid phase extraction (SPE) column 1620, a fluid manifold and valves 1622, and multiple syringe pumps 1624. Example fluid schematic 1700 of the eDNA sensor 1600 is shown in FIG. 17. In this embodiment, the pump 1604 was moved from the back of the filer to the front, transitioning the sensor from a pull configuration to a push configuration, which allows that the pump will not have to be decontaminated between samples, and tubing is easy to sterilize. In addition, the sample inlet may be removed, if a downstream filter can be used between sampling and extraction to protect the valves and tubing. Removing sample inlet may mean that any organic matter in the ocean may be captured, instead of only the material that bypasses the inlet filter.

While the foregoing description has focussed primarily on eDNA sensors and associated methods of detecting eDNA, it will be appreciated that a similar sensor technology and associated methods can be used for detecting environmental RNA (eRNA). In this implementation, RNA's complementary DNA strand is synthesized making a double stranded nucleic acid molecule that can then amplified. RNA is less stable then DNA, so the speed of the protocol would be a factor. However, no significant modifications would be required to be made to the instrument: it would similarly sample, extract, and amplify/detect the RNA present in captured cells, and simply add a reverse transcription step before the amplification with the specific primers. For example, in the case of a qPCR reaction the reverse primer would be used, i.e. the core pumping, valving, thermocycling, fluorescence would be the same. However, it may require different reagents, but could still be done in a single tube.

Also, while the foregoing description has primarily contemplated detection of marine mammals, it will be appreciated that the sensor can be applied to detect other targets, groups of targets, etc. Different genetic primers are designed, specific to that species, genus, family, etc. in the taxonomic structure for identification. The primers are designed such that they do not bind to the incorrect species, but only the target(s). A software can be used to design appropriate primers.

It would be appreciated by one of ordinary skill in the art that the system and components shown in the figures may include components not shown in the drawings. For simplicity and clarity of the illustration, elements in the figures are not necessarily to scale and are only schematic. It will be apparent to persons skilled in the art that a number of variations and modifications can be made without departing from the scope of the invention as described herein.

It is contemplated that any part of any aspect or embodiment discussed in this specification can be implemented or combined with any part of any other aspect or embodiment discussed in this specification.

It should be recognized that features and aspects of the various examples provided above can be combined into further examples that also fall within the scope of the present disclosure.

When used in this specification and claims, the terms “comprises” and “comprising” and variations thereof mean that the specified features, steps or integers are included. The terms are not to be interpreted to exclude the presence of other features, steps or components.

The invention may also broadly consist in the parts, elements, steps, examples and/or features referred to or indicated in the specification individually or collectively in any and all combinations of two or more said parts, elements, steps, examples and/or features. In particular, one or more features in any of the embodiments described herein may be combined with one or more features from any other embodiment(s) described herein.

1. An environmental DNA (eDNA) sensor, comprising:
a sample capture module configured to capture a fluid sample from a marine environment;
a DNA extraction module fluidly coupled to the sample capture module and configured to extract and purify DNA captured by the sample capture module; and
a genetic analysis module fluidly coupled to the DNA extraction module for analyzing the DNA to detect a target species.

2. The eDNA sensor of claim 1, further comprising a microfluidic system configured to control fluid flow in the eDNA sensor and a controller configured to control the microfluidic system and to receive measurements from the genetic analysis module.

3. The method of claim 1, wherein the DNA extraction module and/or the genetic analysis module are implemented on a microfluidic chip.

4. The eDNA sensor of claim 1, further comprising a fluid storage section storing fluids used in the DNA extraction module and the genetic analysis module.

5. The eDNA sensor of claim 1, wherein the sample capture module comprises a sample inlet port through which the fluid sample is received from the marine environment.

6. The eDNA sensor of claim 1, wherein the sample capture module comprises one or more filter membranes for collection of cellular material from the fluid sample.

7. The eDNA sensor of claim 6, wherein the sample capture module further comprises a pump for pumping the fluid sample across the one or more filter membranes for collection of the cellular material.

8. The eDNA sensor of claim 6, wherein the sample capture module further comprises at least one of: a flow sensor configured to track a volume of fluid sampled, and a pressure sensor configured to track material loading on the one or more filter membranes.

9. The eDNA sensor of claim 1, wherein the sample capture module is configured to capture and store a second fluid sample for archival.

10. The eDNA sensor of claim 1, comprising a sensor body housing the DNA extraction module and the genetic analysis module, and wherein the sample capture module is removably coupled to the sensor body.

11. The eDNA sensor of claim 1, wherein the genetic analysis module performs quantitative polymerase chain reaction (qPCR) or DNA sequencing.

12. The eDNA sensor of claim 1, wherein the target species is a marine mammal species.

13. The eDNA sensor of claim 1, further comprising a communication module configured to communicate a result of the genetic analysis module.

14. The eDNA sensor of claim 1, further comprising an on-board power module.

15. An eDNA sensor system, comprising a plurality of the eDNA sensors of claim 1.

16. A method of detecting environmental DNA (eDNA), comprising:
capturing a fluid sample from a marine environment;
automatically extracting DNA from the fluid sample; and
analyzing the DNA to detect a presence of a target species.

17. The method of claim 16, further comprising detecting the presence of the target species, and communicating the presence of the target species to a remote device.

18. The method of claim 16, further comprising updating a density model for the target species based on the presence of the target species in the marine environment.

19. An environmental RNA (eRNA) sensor, comprising:
a sample capture module configured to capture a fluid sample from a marine environment;
an RNA extraction module fluidly coupled to the sample capture module and configured to extract and purify RNA captured by the sample capture module; and
a genetic analysis module fluidly coupled to the RNA extraction module for analyzing the RNA to detect a target species.
